(19) United States
(12) Patent Application Publication
(10) Pub. No.: US 2003/0050693 A1
(43) Pub. Date: Mar. 13, 2003

(54) Minimally invasive delivery system for annuloplasty rings

(76) Inventors: Rodolfo C. Quijano, Laguna Hills, CA (US); Hosheng Tu, Newport Coast, CA (US)

Correspondence Address: Hosheng Tu, 15 Riez, Newport Coast, CA, 92657, US

(21) Appl. No.: 09/949,776

(22) Filed: Sep. 10, 2001

(51) Int. Cl. A61F 2/24

(52) U.S. Cl. 623/2.11; 623/2.36; 606/151; 623/902

(57) ABSTRACT

A delivery system for delivering an annuloplasty ring through a percutaneous intercostal penetration of a patient's chest comprising an elongated handle having a distal end, a distal opening, and a lumen; a ring holder placed within the lumen of the elongated handle and configured to be releasably coupled to the annuloplasty ring, wherein the annuloplasty ring is retractable to a low tubular-profile suitable for being releasably coupled to the ring holder adapted for being placed within said lumen of the handle; and a deployment mechanism coupled to the ring holder and located on the elongated handle, said deployment mechanism being configured for deploying the ring holder out of said distal opening of the handle and for recessing the ring holder within said lumen.

### FIELD OF THE INVENTION

The present invention generally relates to improved medical devices and their use. More particularly, the present invention relates to an annuloplasty or remodeling ring for correction of certain disorders in the heart valves, venous valves, blood vessels or other body conduits for a patient in a minimally invasive manner.

### BACKGROUND OF THE INVENTION

The human's circulatory system consists of a heart and many blood vessels. In its path through the heart, the blood encounters four valves. The valve on the right side that separates the right atrium from the right ventricle has three cusps and is called the tricuspid valve. It closes when the ventricle contracts during a phase known as systole and it opens when the ventricle relaxes, a phase known as diastole. The pulmonary valve separates the right ventricle from the pulmonary artery. The mitral valve, so named because of its resemblance to a bishop's mitre, is in the left ventricle and it separates the left atrium from the ventricle. The fourth valve is the aortic valve that separates the left ventricle from the aorta. In a venous circulatory system, a venous valve is to prevent the venous blood from leaking back into the upstream side so that the venous blood can return to the heart and consequently the lungs for blood oxygenating and waste removing purposes.

In many patients who suffer from diseased or congenitally dysfunctional cardiovascular tissues, a medical implant may be used to correct the problems. A dysfunctional heart valve hinders normal functioning of the atrioventricular orifices and operation of the heart. More specifically, defects such as narrowing of the valve stenosis or defective closing of a valve, referred to as valvular insufficiency, result in accumulation of blood in a heart cavity or regurgitation of blood past the valve. If uncorrected, prolonged valvular insufficiency may cause eventually total valve replacement. On the other hand, certain diseases cause the dilation of the heat valve annulus. Dilation may also cause deformation of the valve geometry or shape displacing one or more of the valve cusps from the center of the valve. Dilation and/or deformation result in an ineffective closure of the valve during ventricular contraction, which results in regurgitation or leakage of blood during contraction.

It is known to use annuloplasty ring in the repair of diseased or damaged atrioventricular valves that do not require replacement. The annuloplasty ring is designed to support the functional changes that occur during the cardiac cycle: maintaining coaptation and valve integrity in systole while permitting good hemodynamics in diastole. The annuloplasty or remodeling ring also provides support for the mitral or tricuspid annulus and restricts expansion of the annulus or portions of the annulus to preset limits. A variety of annuloplasty rings have been employed, ranging from rigid rings of fixed sizes to flexible rings with a degree of adjustability. Obviously, annular prostheses that are of rigid fixed size must be carefully selected and skillfully sutured in place. Thus, an imperfect fit may require corrective surgery to replace the improperly implanted prosthesis. A rigid ring also prevents the normal flexibility of the valve annulus and has a tendency of sutures tearing during the normal movement of the valve annulus. Examples of rigid or partially rigid annuloplasty rings are disclosed in U.S. Pat. No. 5,061,277 and in U.S. Pat. No. 5,104,407.

The annuloplasty ring comprises an inner substrate of a metal such as stainless steel or titanium, or a flexible material such as silicone rubber or Dacron cordage, covered with a biocompatible fabric or cloth to allow the ring to be sutured to the heart tissue. Annuloplasty ring may be used in conjunction with any repair procedures where contracting or stabilizing the valve annulus might be desirable.

Using current techniques, most valve repair, replacement or annuloplasty ring implantation procedures require a gross thoracotomy, usually in the form of a median sternotomy, to gain access into the patient's thoracic cavity. Alternatively, a thoracotomy may be performed on a lateral side of the chest, wherein a large incision is made generally parallel to the ribs, and the ribs are spread apart in the region of the incision to create a large enough opening to facilitate the surgery.

Of particular interest in the present application are techniques for the implantation of an annuloplasty or remodeling ring that can be retracted inside a delivery applicator or canulae for delivering to the desired place. Thereafter the retracted ring is released, expanded, separated from the delivery application, and sutured to the valve annulus with a minimally invasive technique.

Therefore, it would be desirable to provide a delivery system for delivering an annuloplasty ring in a patient's heart comprising a ring holder configured to be releasably coupled to the annuloplasty ring, the ring holder having a plurality of grasping points and an elongated handle for delivering the ring holder with the annuloplasty ring through a percutaneous intercostal penetration of a patient's chest, the handle having a distal opening and a lumen for holding a retracted coupled elements along with a releasably coupled annuloplasty ring during a delivery state. Such a delivery system having a deploying mechanism on the elongated handle configured for deploying the ring holder out of said distal opening of the handle and recessing the ring holder within said lumen of the patient without suffering the above-discussed disadvantages of gross thoracotomy.

### SUMMARY OF THE INVENTION

In general, it is an object of the present invention to provide a retractable annuloplasty ring which may be retracted to be placed inside a lumen of a delivery apparatus used in minimally invasive procedures. It is another object of the present invention to provide a delivery system having the capability for retracting an annuloplasty or remodeling ring to be used in minimally invasive percutaneous procedures. It is still another object of the present invention to provide a method for delivering a retracted annuloplasty ring into place through an intercostal penetration at a patient's chest.

In accordance with one embodiment of the invention, the annuloplasty ring for treating an anatomical annulus comprises a plurality of substantially rigid curved segments that are coupled and adapted to define a circumferential plane, said coupled segments being configured to be placed at least partially circumferentially about the anatomical annulus to form a line to stabilize the anatomical annulus; and a plurality of segment-couplers for securely coupling two adjacent segments at about an outer corner of each segment, wherein said coupled segments are restricted to flex inwardly along the circumferential plane.

At least a portion of the substantially rigid curved segments is covered with a fabric sheath. The fabric sheath may be stretchable or distensible to accommodate the distension of the annuloplasty ring during retracted stage. The fabric sheath may be impermeable to prevent blood from entering into the space of the segments. It may also comprise a silicone layer so that the annuloplasty ring is substantially impermeable to blood or blood components. The silicone layer may be placed between the outer fabric sheath and the inner segments of the annuloplasty ring. The fabric sheath may be suturable to facilitate suturing-in-place of the ring to the surrounding anatomical tissue. The fabric sheath may be made of Dacron or other biocompatible synthetic material.

In another embodiment, the delivery system for delivering an annuloplasty ring through a percutaneous intercostal penetration of a patient's chest comprises an elongated handle having a distal end, a distal opening, and a lumen; a ring holder configured to be releasably coupled to the annuloplasty ring; and a deployment mechanism located on the elongated handle and configured for deploying the ring holder out of the distal opening of the handle and for recessing the ring holder within the lumen. In a preferred embodiment, the annuloplasty ring is retractable to a low tubular-profile suitable for being releasably coupled to the ring holder adapted for being placed within the lumen of the handle.

In a further embodiment, a method for percutaneously delivering an annuloplasty ring for treating an anatomical annulus comprises steps of delivering the retracted annuloplasty ring to the anatomical annulus percutaneously during a delivery phase; deploying the annuloplasty ring during a deployment phase to its fully extended state; and placing and securing the annuloplasty ring at about the anatomical annulus to form a line to stabilize at least a substantial portion of the anatomical annulus.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a top view of a substantially rigid curved segment that is couple-able to another segment for defining an annuloplasty ring configuration of the present invention.

FIG. 2 is a front view, section I-I of a substantially rigid curved segment of FIG. 1.

FIG. 3 is a left-side view, section II-II of a substantially rigid curved segment of FIG. 1.

FIG. 4 is a right-side view, section III-III of a substantially rigid curved segment of FIG. 1.

FIG. 5 is an illustration of two substantially rigid curved segments of FIG. 1 that are coupled together at the secured state of the two segments.

FIG. 6 is an illustration of two substantially rigid curved segments of FIG. 1 that are coupled at its flexing state of the two segments.

FIG. 7 is an annuloplasty or remodeling ring of the present invention comprising a plurality of substantially rigid curved segments that are covered with a fabric cloth.

FIG. 8 is a delivery apparatus comprising an annuloplasty ring holder configured to be releasably coupled to the annuloplasty ring, and an elongated handle for delivering the ring holder and the annuloplasty ring through a percutaneous intercostal penetration of a patient's chest.

FIG. 9 is a delivery apparatus of FIG. 8 at a delivery phase wherein curved segments are coupled and retracted at their flexing state.

FIG. 10 is one embodiment of the deployment mechanism on the elongated handle of the delivery apparatus of FIG. 8.

FIG. 11 is a front end view of the delivery apparatus of FIG. 8, showing elevation of the annuloplasty ring with respect to the axis of the delivery apparatus.

FIG. 12 is an illustrative view of the grasping mechanism for an end piece of the ring holder to grasp an annuloplasty ring.

FIG. 13 is a detailed view of the grasping mechanism for an end piece of the ring holder of FIG. 12.

FIG. 14 is an illustrative view of an annuloplasty ring being delivered to the valve annulus of a patient percutaneously.

### DETAILED DESCRIPTION OF EXEMPLARY EMBODIMENTS

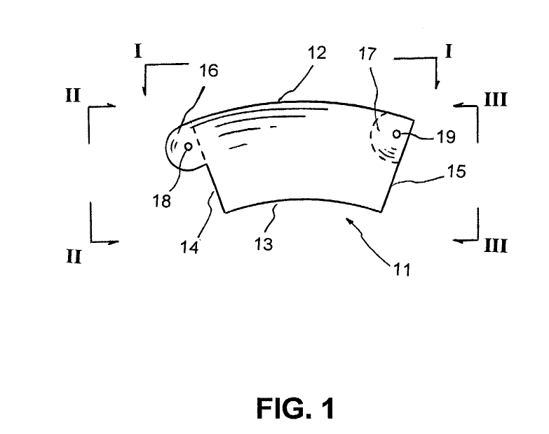

With reference to the drawings FIGS. 1 to 12, what is shown is an embodiment of a retractable annuloplasty ring having extremely low profile adapted for implantation through a delivery apparatus in a minimally invasive percutaneous procedure.

FIG. 1 shows a top view of a substantially rigid curved segment that is coupleable to define an annuloplasty ring of the present invention. An annuloplasty or remodeling ring for treating an anatomical annulus comprises a plurality of substantially rigid curved segments 11, 41, 51 that are coupled to define a circumferential plane, said coupled segments being configured to be placed at least partially circumferentially about the anatomical annulus 91 to form a line to stabilize at least a substantial portion of the anatomical annulus; and a plurality of segment-couplers 18, 19 for securely coupling two adjacent segments at about an outer corners 16, 17 of each segment 11, wherein said coupled segments are generally free to flex with respect to the segment-couplers 18, 19 along said circumferential plane. The “outer comer” of the present invention generally refers to the region adjacent the comer of the segments suitable for coupling the two segments without undue obstruction. The anatomical annulus may comprise a valve annulus or the like.

In one embodiment, the substantially rigid curved segment 11 comprises a right side edge (or line) 15, a left side edge 14, a front side edge 12 and a rear side edge 13. In a preferred embodiment, a first segment-coupler 18 is located at an extended segment comer 16 while a second segment-coupler 19 is located at a recessed segment comer 17 of the representative segment 11. By coupling a plurality of the substantially rigid curved segments, an annuloplasty ring is formed at essentially a circumferential plane. A fabric sheath or cloth is usually wrapped around at least a portion of the segment or segments adapted for suturing onto the annulus tissue. The fabric sheath is preferred stretchable configured for stretching the segments coupled and mounted within the fabric sheath.

For drug therapeutics purposes, the internal space within the fabric sheath of the annuloplasty ring may contain a fluid selected from the group consisting of heparin solution, saline solution, virucidal agents, anti-ulcer agents, anti-inflammatory agents, antibiotics, anti-cancer agents, and their mixture.

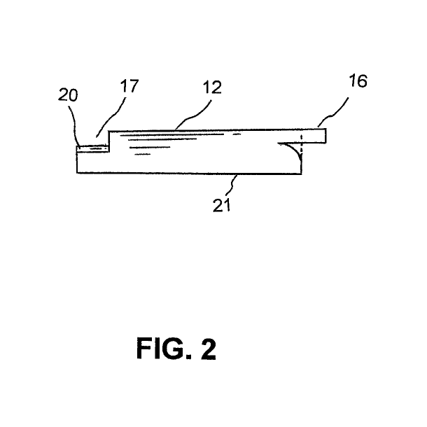

FIG. 2 shows a front view, section I-I of a substantially rigid curved segment 11 of FIG. 1. The substantially rigid curved segment 11 comprises a bottom region 21, wherein the recessed segment comer 17 may further comprise an optional supporting element 20 adapted to improve the flexing movement between the upper extended segment comer 16 and the lower recessed segment comer 17. The supporting element 20 could be a gasket-type or washer-type flat element made of biocompatible silicone, polyurethane, polyester, or Teflon material.

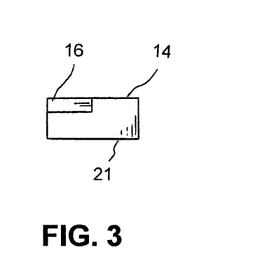

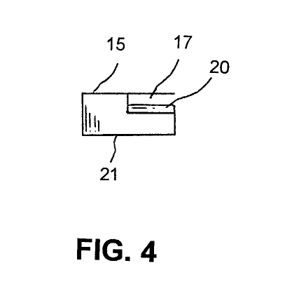

FIG. 3 shows a left-side view, section II-II of a substantially rigid curved segment 11 of FIG. 1, whereas FIG. 4 shows a right-side view, section III-III of a substantially rigid curved segment 11 of FIG. 1. The left-side edge 14 of a first segment is couple-able to the right-side edge of a second segment and so forth to form an annuloplasty ring.

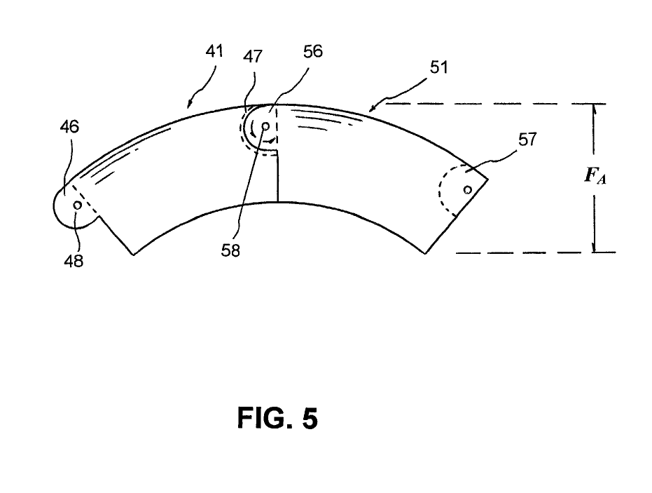

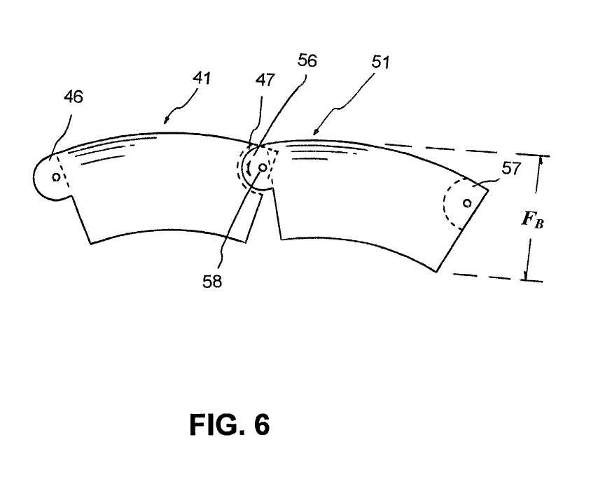

FIG. 5 and FIG. 6 show an illustration of two substantially rigid curved segments 41, 51 that are coupled together at its secured state and flexing state, respectively. Here the “secured state” of any two coupled segments of the present invention is defined as the two segments being coupled securely, but difficult to flex, with respect to the segment-coupler 58 so that the right-side edge of a left-side segment matches intimately and securely with the left-side edge of a right-side segment of these two coupled segments. The methods for securing the two segments at its secured state may be selected from a group consisting of latching, locking, magnetic attraction, bonding and the like. When two segments are at their secured state, the segments are restricted to flex inwardly along the circumferential plane.

The “flexing state” of any two coupled segments is further defined in the present invention as the two segments being coupled but free to flex with respect to the coupling axis at the segment-coupler 58 in both directions along the circumferential plane. Any two coupled segments are free to flex at the flexing state until the right-side edge of a left-side segment is secured to the left-side edge of a right-side segment at the secured state.

The segment-coupler 18, 19, 58, 59 may be a hinge structure configured for restricting said coupled segments to flex inwardly, wherein the hinge structure may optionally be a loaded spring adapted for restricting the coupled segments to flex inwardly. The segment-coupler 18, 19 may further comprise a securing mechanism adapted for securing the two coupled segments in a secured state.

The coupled segments have generally a “tubular-profile” that is defined in the present invention as the maximum profile circumferentially to fit inside a lumen of a tubular delivery applicator. A higher tubular-profile implies that a larger lumen is needed to hold the coupled segments axially. For a minimally invasive procedure to introduce the applicator through a percutaneous intercostal penetration of a patient's chest, a low tubular-profile is desirable. The substantially rigid curved segment 11 may be selected from the group consisting of Nitinol, Nickel-Titanium alloy, stainless steel, biocompatible metal, biocompatible plastic, and the like.

For illustration purposes, the tubular-profile for a coupled two-segment unit as shown in FIG. 5 is about F_{A} (or π×F_{A}) at a secured state. To facilitate minimally invasive percutaneous delivery procedures, the coupled two-segment unit is flexed to a lower tubular-profile F_{B} (or π×F_{B}) at a flexing state as shown in FIG. 6. The coupled segments of the annuloplasty ring of the present invention are to be flexed to the lowest tubular-profile for the annuloplasty ring and configured to be retracted into a lumen of the delivery apparatus during the delivery phase. As illustrated in the present invention, a retractable annuloplasty or remodeling ring of the present invention may comprise any annuloplasty ring that has a tubular-profile less than its corresponding tubular-profile of the fully deployed state. For example, the tubular-profile of a retracted annuloplasty ring could be less than one-half of that of the non-retracted annuloplasty ring, though more reduction on tubular-profile for a ring retraction is generally favorable. In an alternate embodiment, the annuloplasty ring of the present invention may comprise a ring that is made of flexible retractable material. The “fully deployed state” in the present invention means the annuloplasty ring is in its functional configuration suitable for suturing onto the anatomical tissue.

The annuloplasty ring is retractable to a low tubular-profile suitable for being releasably coupled to the ring holder adapted for being placed within said lumen of the handle, wherein a method for retracting the annuloplasty ring to a low tubular-profile may comprise compressing the ring inwardly or pulling/bending the ring outwardly. The method of compressing the ring inwardly or pulling/bending the ring outwardly so that the annuloplasty ring is retracted to be placed inside the lumen of a delivery apparatus is well known to an ordinary person who is skilled in the art. In one embodiment, the method for compressing the ring inwardly is to compress about one end of the ring against the opposite end of the ring resulting in reduced tubular-profile. In another embodiment, the method for pulling/bending the ring outwardly is to hold about a first end of the ring and pull the opposite end of the ring away from the first end resulting in reduced tubular-profile.

Northrup, III et al. in U.S. Pat. No. 5,961,539 discloses an apparatus for sizing, stabilizing, and/or reducing the circumference of an anatomical structure, entire contents of which are incorporated herein by reference. More particularly Northrup. III et al. discloses a delivery system for delivering a plurality of substantially rigid suture support segments during a surgical procedure, the substantially rigid suture support segments to be placed circumferentially about an anatomical structure for stabilizing the anatomical structure along the line of discrete suture support segments, the delivery system comprising a plurality of segment holders, the plurality of segment holders being readily releasably securable to the plurality of substantially rigid suture support segments and separating the plurality of substantially rigid suture support segments from each other. The plurality of segment holders comprise linking structure to link the segment holders together, whereas the segment holders, when linked together, define consistent dimensions to precisely size an anatomical vascular structure and define consistent intervals for delivering the suture support segments to an anatomical vascular structure. However, the plurality of segment holders with linking structure are fee to flex the suture support segments at both directions, but does not provide the segments at a “secured state” as disclosed by the present invention.

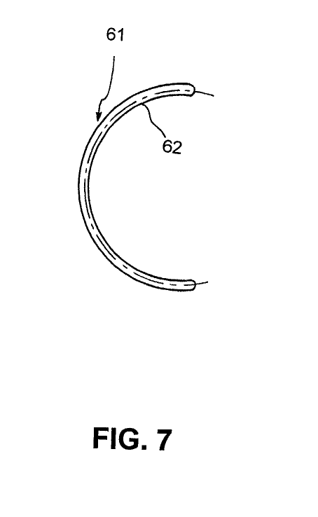

FIG. 7 shows an annuloplasty or remodeling ring 61 of the present invention comprising a plurality of substantially rigid curved segments that are covered with a fabric cloth 62. The annuloplasty ring to be retractably used in the present invention may be split or continuous, and may have a variety of shapes, including circular, D-shaped, C-shaped, or kidney-shaped. Examples are seen in U.S. Pat. Nos. 4,917,698, 5,061,277, 5,290,300, 5,350,420, 5,104,407, 5,064,431, 5,201,880 and 5,041,130, which are all incorporated herein by reference.

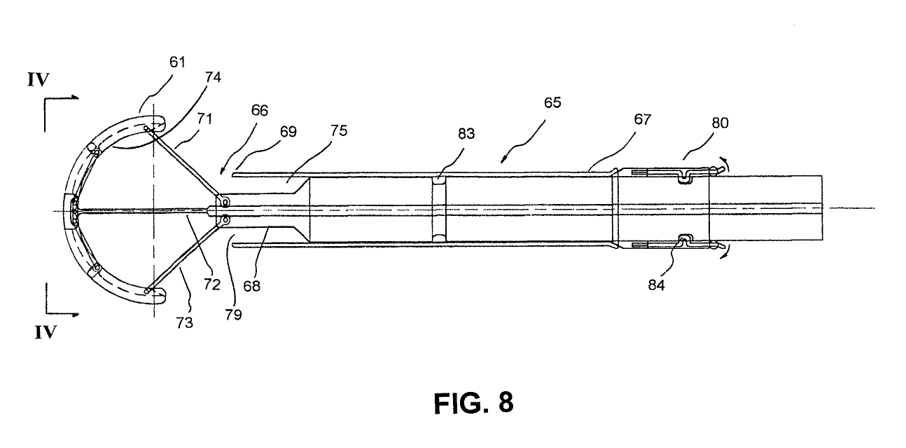

FIG. 8 shows a delivery apparatus 65 comprising an annuloplasty ring holder 66 configured to be releasably coupled to the annuloplasty ring 61, and an elongated handle 67 for delivering the ring holder with the annuloplasty ring through a percutaneous intercostal penetration of a patient's chest. The ring holder 66 further comprises a holder base 68 and a plurality of holder members 71, 72, 73 that are connected to the holder base and extendable out of the distal end 69 of the delivery apparatus 65.

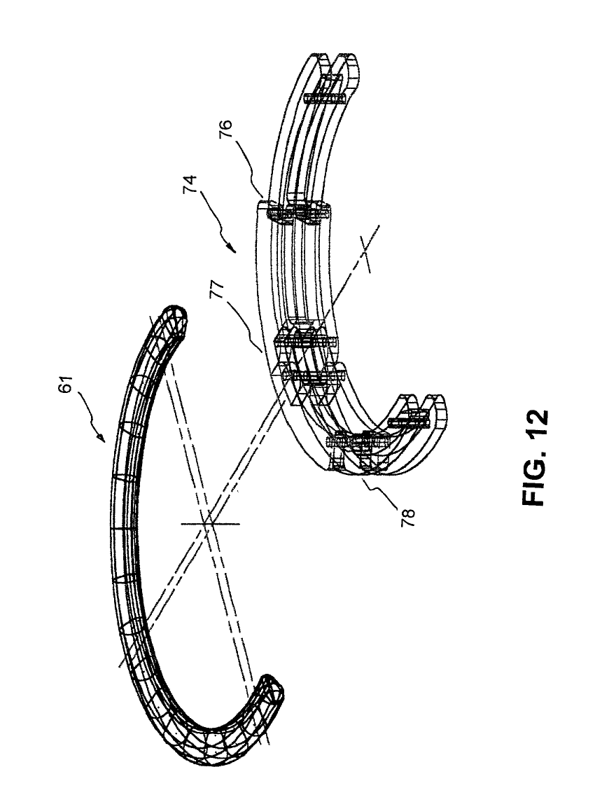

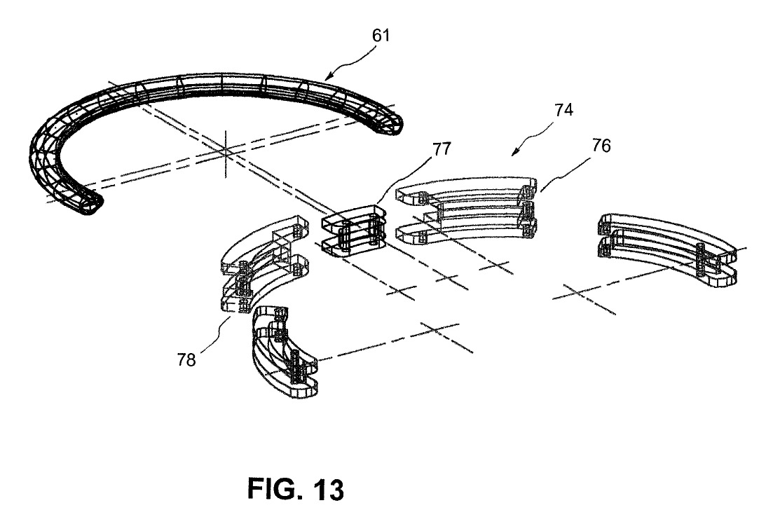

As shown in FIG. 12, the ring holder 66 further comprises an end piece 74 adapted for grasping the annuloplasty ring 61 at a plurality of points 76, 77, 78 (the grasping points), the end piece 74 being attached to the holder members 71, 72, 73. The end piece 74 may be configured to having a plurality of end-piece members that are defined to be between any two adjacent grasping points. For example, a first end-piece member is the section of the end piece between grasping points 71 and 72. A second end-piece member is the section of the end piece between grasping points 72 and 73; and so forth. Each end-piece member is relatively rigid. There is a flexing joint between any two adjacent end-piece members. FIG. 13 shows a detailed view of the grasping mechanism for an end piece 74 of the ring holder of FIG. 12. The structure for the end piece of the presentation as shown in FIG. 13 is well known to one ordinary person who is skilled in the art.

The end-piece members are generally flexible and the flexing movement is controlled by the holder members 71, 72, 73 according to the deployment mechanism 80. Therefore, a conventional annuloplasty ring or the annuloplasty ring of the present invention may be releaseably attached to the end piece 74 and form a retracted structure for being placed within a lumen 75 of the delivery apparatus 65.

The ring holder 66 of the delivery system may comprise means for grasping the annuloplasty ring on the ring holder and releasing the annuloplasty ring from the ring holder when said ring is deployed at about a valve annulus of a patient. In one embodiment, the grasping means may comprise at least one suture for fastening said ring to the ring holder. The suture is eventually separated from the ring holder after the ring is in place.

The handle 67 has a distal opening 79 at its distal end 69 and a lumen 75 for holding a retracted holder members 71, 72, 73 along with a releasably coupled annuloplasty ring 61 during a delivery phase. One type of the deployment mechanism 80 on the elongated handle 67 is configured either for deploying the holder members 71, 72, 73 of the ring holder 66 out of the distal opening 79 of the handle or for recessing the holder members of the ring holder within said lumen 75.

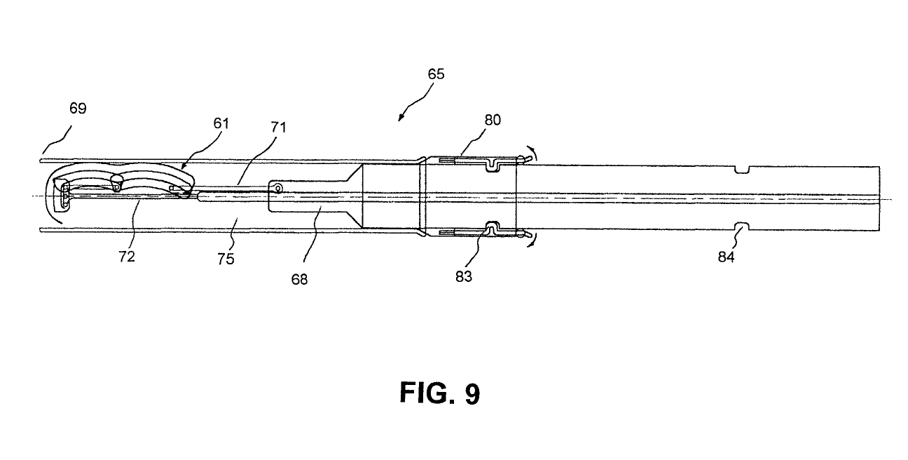

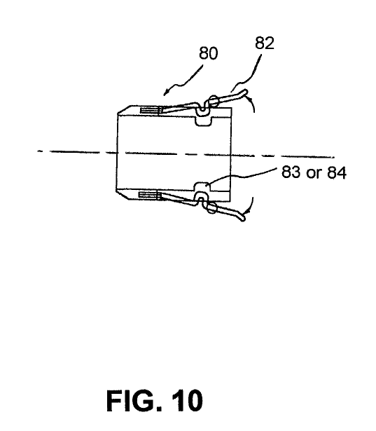

FIG. 9 shows the delivery apparatus 65 at a delivery phase wherein the curved segments 11 of the annuloplasty ring 61 are coupled and retracted at their flexing state. The annuloplasty ring under the retracted phase is at about its minimal tubular-profile suitable for the annuloplasty ring 61 to stay comfortably within the lumen 75 of the delivery apparatus 65. For illustration purposes, FIG. 10 shows a deployment mechanism 80 on the elongated handle 67 of the delivery apparatus 65, wherein the deployment mechanism comprises a spring-loaded clutch 82. The clutch 82 may be loosened and locked in at a first latch 83 on the handle for retracting the annuloplasty ring. Further, the clutch 82 may be loosened and locked in at a second latch 84 on the handle for deploying the annuloplasty ring. Other available deployment mechanisms are equally applicable.

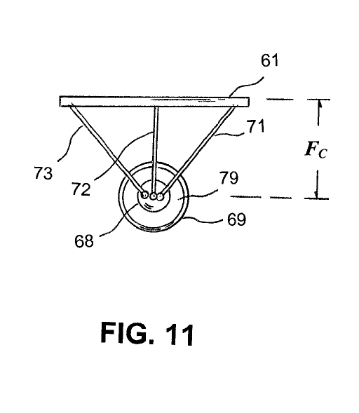

FIG. 11 shows a front end view of the delivery apparatus 65 of FIG. 8, showing elevation of the annuloplasty ring with respect to a longitudinal axis of the delivery apparatus. In one embodiment, the annuloplasty ring is at a different plane with respect to a longitudinal axis of the delivery apparatus when the annuloplasty ring 61 is deployed to a longitudinal axis of the delivery apparatus when the annuloplasty ring 61 is deployed out of the lumen 79 of the delivery apparatus 65. The elevation is characterized by a distance F_{C}, wherein the annuloplasty ring 61 is at the same plane when F_{C} is zero. The angle of the holder members 71, 72, 73 with respect to the longitudinal axis of the delivery apparatus 65 may be from 0 to about 90 degrees configured for the annuloplasty ring to be deployed at an appropriate position suitable for placing the ring at least partially circumferentially about the anatomical annulus to stabilize the anatomical annulus. The holder members 71, 72, 73 of the ring holder is delivered out of said distal opening 79 of the handle at an angle with respect to a longitudinal axis of the handle 67.

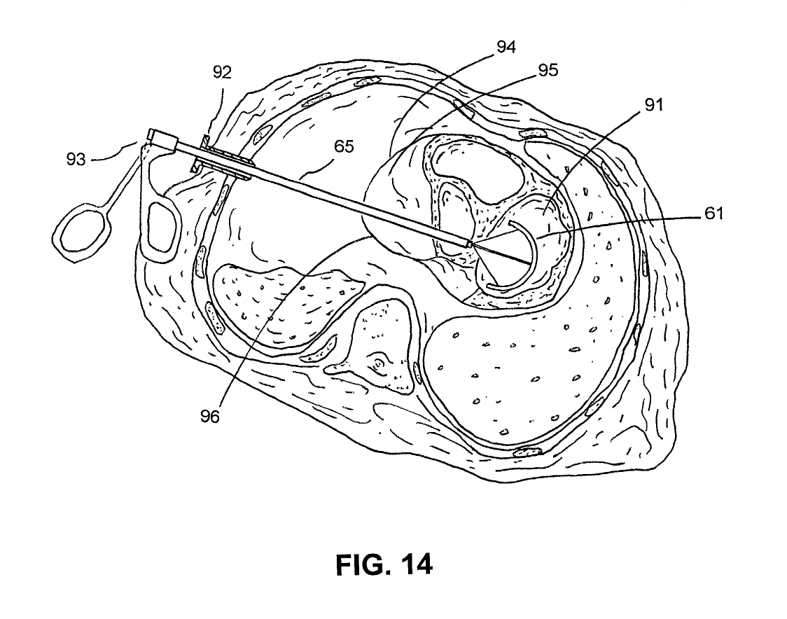

FIG. 14 shows an illustrative view of an annuloplasty ring 61 being delivered to the valve annulus 91 of a patient, wherein the patient's heart 94 is positioned to show left atrium 95 and atrium wall 96. In operation, a delivery apparatus 65 of the present invention having an alternate deployment mechanism 93 at the proximal end of the apparatus 65 is deployed through an intercostal penetration. The annuloplasty ring may be introduced through a cannula or trocar 92 positioned in one of percutaneous intercostal penetrations, the cannula or trocar having a proximal end disposed outside of the patient and a distal end disposed within the chest. A general minimally invasive procedure is well known to the ordinary clinicians who are skilled in the art. Examples are U.S. Pat. Nos. 5,972,030 and 5,613,937 to Garrison et al., entire contents of which are incorporated herein by reference.

In a minimally invasive procedure, a method for percutaneously delivering an annuloplasty ring for treating an anatomical annulus may comprise the steps of: (1) delivering said annuloplasty ring to the anatomical annulus percutaneously during a delivery phase; (2) deploying the annuloplasty ring during a deployment phase; and (3) placing and securing the annuloplasty ring at about the anatomical annulus. The method may further comprise forming a plurality of percutaneous intercostal penetrations in a patient's chest, each of the percutaneous intercostal penetrations being within an intercostal space between two adjacent ribs, wherein said annuloplasty ring is delivered through one of the percutaneous intercostal penetrations. The annuloplasty ring may be introduced through a cannula or trocar positioned in one of percutaneous intercostal penetrations, the cannula or trocar having a proximal end disposed outside of the patient and a distal end disposed within the chest.

In a minimally invasive procedure, the method may further comprise sizing a patient's valve annulus by means of a sizing instrument introduced through one of said plurality percutaneous intercostal penetrations and through an internal penetration, wherein said internal penetration is formed through a wall of a patient's heart using a cutting tool introduced through one of said percutaneous intercostal penetrations in the patient's chest.

In one embodiment, a method for percutaneously delivering an annuloplasty ring for treating an anatomical annulus is when a patient's heart is arrested.

In another embodiment, the minimally invasive method may further comprise viewing the patient's heart through one of said percutaneous intercostal penetrations by means of using an endoscope positioned through one of said percutaneous penetrations. Example for sizing a patient's valve annulus by means of sizing instrument and for viewing the patient's heart by means of using an endoscope through one of the percutaneous penetrations is disclosed in U.S. Pat. No. 5,613,937 to Garrison et al., entire disclosure of which is incorporated herein by reference.

From the foregoing description, it should now be appreciated that a retractable annuloplasty ring and a delivery system for delivering the retractable annuloplasty ring in a minimally invasive manner percutaneously have been disclosed for implantation in a heart valve annulus. While the invention has been described with reference to a specific embodiment, the description is illustrative of the invention and is not to be construed as limiting the invention. Various modifications and applications may occur to those who are skilled in the art, without departing from the true spirit and scope of the invention, as described by the appended claims.

What is claimed is:

1. A delivery system for delivering an annuloplasty ring through a percutaneous intercostal penetration of a patient's chest comprising:
an elongated handle having a distal end, a distal opening, and a lumen;
a ring holder placed within the lumen of the elongated handle and configured to be releasably coupled to the annuloplasty ring, wherein the annuloplasty ring is retractable to a low tubular-profile suitable for being releasably coupled to the ring holder adapted for being placed within said lumen of the handle; and
a deployment mechanism coupled to the ring holder and located on the elongated handle, said deployment mechanism being configured for deploying the ring holder out of said distal opening of the handle and for recessing the ring holder within said lumen.

2. The delivery system of claim 1, wherein said low tubular-profile of a retracted annuloplasty ring is less than half of the tubular-profile of the non-retracted annuloplasty ring.

3. The delivery system of claim 2, wherein a method for retracting said annuloplasty ring to a low tubular-profile comprises either compressing the ring inwardly or pulling and bending the ring outwardly.

4. The delivery system of claim 2, wherein the ring holder is delivered out of said distal opening of the handle at an angle with respect to a longitudinal axis of the handle.

5. The delivery system of claim 1, wherein the ring holder further comprises means for grasping the annuloplasty ring onto the ring holder and releasing the annuloplasty ring from the ring holder when said ring is deployed at about a valve annulus of a patient, the grasping means comprising at least one suture for fastening said ring onto the ring holder.

6. The delivery system of claim 1, wherein the ring holder further comprises means for grasping the annuloplasty ring on the ring holder and releasing the annuloplasty ring from the ring holder when said ring is deployed at about a valve annulus of a patient, the grasping means comprising a plurality of grasping points for fastening said ring onto the ring holder at said grasping points.

7. The delivery system of claim 1 further comprising a cannula or a trocar positioned in the percutaneous intercostal penetration, the cannula having a proximal end disposed outside of the patient's heart and a distal end disposed within a patient's chest.

8. An annuloplasty or remodeling ring for treating an anatomical annulus comprising a plurality of substantially rigid curved segments that are coupled and adapted to define a circumferential plane, said coupled segments being configured to be placed at least partially circumferentially about the anatomical annulus to form a line to stabilize the anatomical annulus; and a plurality of segment-couplers for securely coupling two adjacent segments at about an outer comer of each segment, wherein said coupled segments are restricted to flex inwardly along said circumferential plane.

9. The annuloplasty or remodeling ring of claim 8, wherein the anatomical annulus comprises a valve annulus.

10. The annuloplasty or remodeling ring of claim 8, wherein said segment-coupler is a hinge structure configured for flexing said coupled segments relative to each other.

11. The annuloplasty ring or remodeling ring of claim 10, wherein said segment-coupler further comprises a securing mechanism adapted for securing the two coupled segments in a secured state.

12. The annuloplasty or remodeling ring of claim 10, where the hinge structure further comprises a loaded spring adapted for restricting said coupled segments to flex relative to each other in one direction.

13. The annuloplasty or remodeling ring of claim 8, wherein at least a portion of said segments is covered with a fabric sheath.

14. The annuloplasty or remodeling ring of claim 13 further comprising a plurality of sutures passing through said fabric sheath configured for attaching said annuloplasty or remodeling ring onto the anatomical annulus.

15. A method for percutaneously delivering an annuloplasty ring for treating an anatomical annulus, the annuloplasty ring comprising a plurality of substantially rigid curved segments that are coupled and adapted to define a circumferential plane, said coupled segments being configured to be placed at least partially circumferentially about the anatomical annulus to form a line to stabilize the anatomical annulus; and a plurality of segment-couplers for securely coupling two adjacent segments at about an outer comer of each segment, wherein said coupled segments are restricted to flex inwardly along said circumferential plane;
the method comprising steps of:
delivering said annuloplasty ring to the anatomical annulus percutaneously during a delivery phase;
deploying the annuloplasty ring during a deployment phase; and
placing and securing the annuloplasty ring at about the anatomical annulus.

16. The method of claim 15 further comprising forming a plurality of percutaneous intercostal penetrations in a patient's chest, each of the percutaneous intercostal penetrations being within an intercostal space between two adjacent ribs, wherein said annuloplasty ring is delivered through one of the percutaneous intercostal penetrations.

17. The method of claim 16 further comprising sizing a patient's valve annulus by means of a sizing instrument introduced through one of said plurality percutaneous intercostal penetrations and through an internal penetration, wherein said internal penetration is formed through a wall of a patient's heart using a cutting tool introduced through one of said percutaneous intercostal penetrations in the patient's chest.

18. The method of claim 16, wherein the annuloplasty ring is introduced through a cannula or trocar positioned in one of percutaneous intercostal penetrations, the cannula or trocar having a proximal end disposed outside of the patient and a distal end disposed within the chest.

19. The method of claim 15, wherein a patient's heart is arrested.

20. The method of claim 16 further comprising viewing the patient's heart through one of said percutaneous intercostal penetrations by means of using an endoscope positioned through one of said percutaneous penetrations.
